(19) United States
(12) Patent Application Publication
(10) Pub. No.: US 2008/0011310 A1
(43) Pub. Date: Jan. 17, 2008

(54) EXTERNAL PENILE COMPRESSION CUFF WITH ABSORBENT POUCH

(75) Inventors: David W. ANDERSON, Brooklyn Park, MN (US); Gerald W. Timm, Minneapolis, MN (US)

Correspondence Address: HAMRE, SCHUMANN, MUELLER & LARSON, P.C., P.O. BOX 2902, MINNEAPOLIS, MN, 55402-0902, US

(73) Assignee: GT Urological, LLC, Minneapolis, MN (US)

(21) Appl. No.: 11/742,232

(22) Filed: Apr. 30, 2007

Related U.S. Application Data
(60) Provisional application No. 60/796,946, filed on May 3, 2006.

(51) Int. Cl. A61F 5/48; A61M 1/00

(52) U.S. Cl. 128/885; 604/322

(57) ABSTRACT

A penile compression device for preventing urinary leaking and method for the same are described. The penile compression device includes a cuff component and an absorbent component. The penile compression device compresses the penis and imparts an occlusive effect on the urethra. A result of this compression is the control of urine leakage in men suffering from urinary stress incontinence. Urethral compression may be concentrated on the urethra through penile compression device, where the device may be opened for penile insertion between a smaller (compressive) configuration and a larger (non-compressive) configuration. The absorbent component can capture and hold any inadvertent urine that may leak past the cuff component.

### CROSS REFERENCE TO RELATED APPLICATIONS

The present application claims the benefit of U.S. Provisional Patent Application Ser. No. 60/796,946, filed May 3, 2006, and entitled “External Penile Compression Cuff with Disposable Absorbent Pouch”, which is incorporated herewith by reference in its entirety.

### FIELD

The technical disclosure herein relates to a penile compression device. More particularly, this disclosure relates to an external penile compression cuff and method for the same that externally compresses the penis to occlude the urethra and prevent or at least significantly reduce urinary leakage.

### BACKGROUND

Penile compression clamps are well known and widely used to prevent urine leakage. In particular, penile clamps have been applied externally onto the penis for clamping the same. Such external penile clamps have been produced both as long-term reusable devices and also as short-term devices limited to one-week use. Typically, such devices have been employed for use in the inspection and treatment of diseases, wounds and other abnormal conditions of the bodies of humans and lower animals.

For example, Bard, Inc. produces the Cunningham clamp. The Cunningham clamp provides two pivoting arms connected by a single-hinge. Clamp arm surfaces that face the penis are padded for intended comfort. The Cunningham employs a ratcheted latch to clamp the two arms closer together and compress the penis. This clamp is reported to be reusable, however, the large clamping forces often lead to pain, swelling, and penile skin break down.

The Squeezer Klip™ disclosed in U.S. Pat. No. 6,463,932 provides a padded top and bottom arm in a pivoting configuration. Like the Cunningham, the Squeezer Klip™ is a hinged, reusable clamp. The dorsal and ventral arms include pressure-applying projections intended to contact the urethra, and preferentially apply pressure between the dorsal veins and arteries. The Squeezer Klip™ avoids direct compression of the neurovascular bundle. Patients are often incapable, however, of repeatedly applying the pressure-applying projections to the appropriate location to effect urethral closure. A screw mechanism clamps the penis using a fine compression adjustment via a threaded adjustment knob. The screw mechanism, however, requires a higher degree of manual dexterity to adjust compression, which can be difficult for older arthritic men.

The C3 clamp disclosed in U.S. Pat. No. 5,184,629 provides a clamp intended for use that is limited to approximately one week. The C3 is constructed from a co-extruded and thermoformed polyolefin sheet. The resultant component has two clam shelled halves connected by a hinge. The penis is placed through a portal between the two halves and the halves are folded over to compress the penis. The clam shells are held closed by a Velcro® strap. Force is localized on the urethra by presence of a raised bump on the bottom clam shell half. The C3 is available in only two fixed sizes. As there is great penile anatomical variation requiring multiple clamp sizes, a clamp frequently may be improperly selected. Further, the ability to operate the strapping system is often difficult for older, arthritic men. Such inconsistencies in sizing and the user dependent strapping system have made the C3 less reliable in its ability to control leakage.

None of the clamps above entirely eliminated urinary leakage.

Other clamps have been disclosed in U.S. Pat. Nos. 6,609,522, 6,289,895 B1, 6,609,522 B2, and 6,234,174 B1. These patents, however, provide detailed straps, latches, hinges and mechanisms for “dialing” in required urethral pressure. Experience with the C3 and such clamps indicate that users may be confused by the added complexity of such components.

Experience with such penile clamps indicates that urine leakage still occurs despite penile compression and urethral occlusion. This leakage is due to clamp movement resulting from bodily movement and rubbing against undergarments. Leakage is also likely to occur during placement and removal of the clamp from the penis. As a result, it has been disclosed in a previous application of GT Urological, LLC, the Assignee of the instant application, to use an absorbent attachment as described U.S. patent application Ser. No. 11/122,395 for “Penile Compression Device,” which is now published as U.S. Patent Application Publication No. 2005-0256365 having the same title.

While such clamps and devices as described above provide some advancement for controlling urinary dysfunction and protecting against bladder malfunction, improvements may yet be made on urethral compression devices for males experiencing stress urinary incontinence. There is a further need for a device that provides optimum comfort and that is easy to apply and remove, while sufficiently preventing urinary leakage. Improvements may still be made to a device for urethral compression that provides an absorbent mechanism that can be conveniently and comfortably attached to the compression device.

### SUMMARY

It is a purpose of the technical disclosure herein to overcome such difficulties described above, thereby providing an improved penile compression device. The penile compression device described herein provides a cuff structure that can compress the penis and the urethra. In the compression position, the cuff structure can sufficiently impart compression on the male urethra to occlude the urethra and prevent urinary leakage. The penile compression device described herein also includes an absorbent structure that can be conveniently and comfortably attached to the cuff structure. The absorbent structure can absorb and hold any inadvertent urine leakage.

Generally, a penile compression device includes a cuff component and an absorbent component attached to the cuff component. In one embodiment, the cuff component is adapted to compress a penis and impart compression on the urethra of the penis, to press a portion of the absorbent component against the penis when the cuff component compresses the penis, and to occlude the urethra when the cuff component compresses the penis. The cuff component and the absorbent component prevent urine leakage and spillage from the urethra and penis.

In one embodiment, the cuff component is adapted to surround the penis and to apply an even circumferential compression on the penis.

In another embodiment, the cuff component is adapted to apply a localized compression on the penis. In yet another embodiment, the cuff component is adapted to apply a localized compression of the penis by activating a hydraulic compression mechanism to expand for example an occluding member.

In yet another embodiment, the cuff component is adapted to compress the penis when the cuff component is manually tightened, or when an electro-mechanical compression mechanism is activated to tighten the cuff component.

In yet another embodiment, the cuff component is intrinsically biased toward a compressive position. The cuff component is configured to be expanded in a non-compressive position so as to be adapted to allow placement on or removal from the penis. The cuff component is configured to return to the compressive position so as to be adapted to allow placement on and compression of the penis.

In one embodiment of an absorbent component, the absorbent component is configured to be removably attached or permanently attached to the cuff component.

In another embodiment, the absorbent component is constructed and arranged to allow a portion of the penis to not be wrapped by the absorbent component.

In one embodiment of a method of externally compressing a penis, a cuff component and an absorbent component are applied onto a penis. The step of applying the cuff component onto the penis includes compressing the penis and imparting compression on the urethra of the penis. The step of applying the cuff component and the absorbent component onto the penis includes pressing a portion of the absorbent component against the penis. The urethra is then occluded when the cuff component imparts a compression on the urethra.

In one embodiment, the step of applying the cuff component on the penis includes surrounding the penis, and the step of compressing the penis includes one of applying an even circumferential compression on the penis or applying a localized compression on the penis.

In one embodiment, the step of applying the cuff component onto the penis includes one of manually tightening the cuff component or electromechanically tightening the cuff component. In yet another embodiment, the step of applying the cuff component onto the penis includes expanding the cuff component, from an intrinsically contracted compressive position to an expanded non-compressive position so as to allow placement on or removal of the cuff component from the penis, and returning the cuff component to the compressive position so as to allow compression of the penis.

The penile compression device of the present application provides suitable neurovascular load distribution that can also impart localized compression on the urethra. The penile compression device can be universally fitting, while improving user comfort and urine leakage prevention effect.

These and other various advantages and features of novelty are pointed out in the following detailed description. For better understanding of the technical disclosure, its advantages and benefits obtained by its use, reference should also be made to the drawings which form a further part hereof, and to accompanying descriptive matter, in which there are illustrated and described specific examples of an apparatus in accordance with inventive concepts.

### BRIEF DESCRIPTION OF THE DRAWINGS

Like reference numbers generally indicate corresponding elements in the Figures. The embodiments illustrated are exemplary only and are in accordance with the inventive concepts disclosed herein.

FIG. 1A is a perspective view of one embodiment for a penile compression device. The device is shown before compression of the penis.

FIG. 1B is a perspective view of the penile compression device shown in FIG. 1A. The device is shown applied to the penis and the device is in a position where the penis is compressed.

FIG. 2A is a side view of another embodiment for a penile compression device. The device is shown in a compression position, where the penis is compressed.

FIG. 2B is an end view of the penile compression device of FIG. 2A.

The device is shown in the compression position.

FIG. 2C is another end view of the penile compression device shown in

FIG. 2A. The device is shown in a position before compression.

FIG. 3A is a perspective view of another embodiment for a penile compression device. The device is shown in a position before use for compression of a penis.

FIG. 3B is another perspective view of the penile compression device of FIG. 3A. The device is shown in a compression position when it may be used for compression of a penis.

FIG. 3C is another perspective view of the penile compression device of FIG. 3A, except that the device further shows one embodiment of a urethral occlusive member that may be incorporated with the device.

FIG. 3D is an end view of the penile compression device of FIG. 3C having the urethral occlusive member incorporated. The device is shown in a position before compression of a penis.

FIG. 3E is another end view of the penile compression device of FIG. 3C having the urethral occlusive member incorporated. The device is shown in a position, where a penis is inserted in the open end region of the device and the penis and urethra are shown compressed.

FIG. 4A is a perspective view of yet another embodiment for a penile compression device.

FIG. 43 is a partially exploded perspective view of the penile compression device of FIG. 4A.

FIG. 5A is an end view of yet another embodiment for a penile compression device. The device is shown in a position before use for compression of a penis.

FIG. 5B is another end view of the penile compression device of FIG. 5A. The device is shown in a compression position, for when it may be used to compress a penis.

FIG. 6 is a schematic view of one embodiment of an electro-mechanical compression mechanism, which may be employed in the device of FIGS. 5A and 5B.

FIG. 7A is an end view of yet another embodiment for a penile compression device. The device is shown in a position before use for compression of a penis.

FIG. 7B is another end view of the penile compression device of FIG. 7A. The device is shown in a compression position, for when it may be used to compress a penis.

FIG. 8 is a schematic view of one embodiment of an electro-hydraulic compression mechanism, which may be employed in the device of FIGS. 7A and 7B.

FIG. 9A is a perspective view of a known penile compression clamp.

FIG. 9B is an end view of the penile compression clamp of FIG. 9A. The clamp is shown in use while compressing a penis and urethra.

FIG. 10A is a perspective view of one embodiment of an absorbent structure which may be used in a penile compression device. The absorbent structure is shown where it can be used with the clamp of FIGS. 9A and 9B as one example. The absorbent structure is shown before use for compressing a penis.

FIG. 10B is another perspective view of the absorbent structure of FIG. 10A. The absorbent structure is shown in use when compressing a penis.

FIG. 11A is a perspective view of another embodiment of an absorbent structure which may be used in a penile compression device. The absorbent structure is shown where it can be used with the clamp of FIGS. 9A and 9B as one example. The absorbent structure is shown before use for compressing a penis.

FIG. 11B is another perspective view of the absorbent structure of FIG. 11A. The absorbent structure is shown in use when compressing a penis.

FIG. 12A is a side view of one embodiment of a pressure transducer used with a penile compression device.

FIG. 12B is a side view of another embodiment of a pressure transducer used with a penile compression device.

FIG. 13 is a side view of one embodiment of a sensor component used with a penile compression device.

FIG. 14 is an end sectional view of one embodiment of an absorbent component construction.

FIG. 15 is an end sectional view of another embodiment of an absorbent component construction.

### DETAILED DESCRIPTION

A penile compression device is described herein that provides a cuff structure, which can compress the penis and the urethra from an initial rest position of the device to a compression position. In the compression position, the devices described herein can sufficiently compress the male urethra to prevent or at least significantly reduce urinary leakage. A penile compression device as described herein can provide improved comfort, and that can be easy to apply and remove. Further, a penile compression device as described herein can provide a convenient and comfortable absorbent mechanism.

Generally, a penile compression device as described herein includes a cuff component and an absorbent component attached to the cuff component.

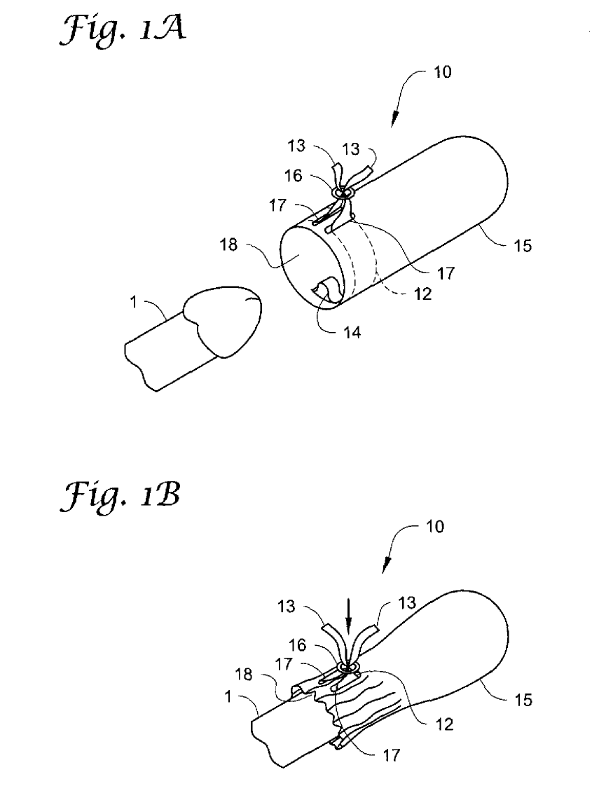

FIGS. 1A and 1B illustrate one embodiment of a penile compression device 10. The penile compression device 10 includes a cuff component 12 and an absorbent component 15. The penis 1 may be inserted through the cuff component 12 through an open end or open region 18 defined by the cuff component 12 and the absorbent component 15.

The cuff component 12 is constructed as a securing member having free ends 13 that can be brought together or proximate of each other in a generally loop-like configuration. The generally loop-like configuration is configured so that a penis can be inserted through the “loop” and is capable of being tightened, or the opening therein is further closed, when compression of the penis and urethra is desired. A retaining member 16 can hold portions of the securing member of the cuff component 12 together, and particularly portions of the securing member proximate the free ends 13.

As shown in FIG. 1A, an occlusive protrusion 14 can be disposed on a surface of the securing member of the cuff component 12, such that the occlusive protrusion 14 faces inward when the securing member is in the generally loop-like configuration. The occlusive protrusion 14 is configured so that it can be positioned to contact the underside of the penis 1. In one embodiment, the occlusive protrusion 14 can be sized and configured to impart localized compression on the penis and occlude the urethra when the compression device 10 is used.

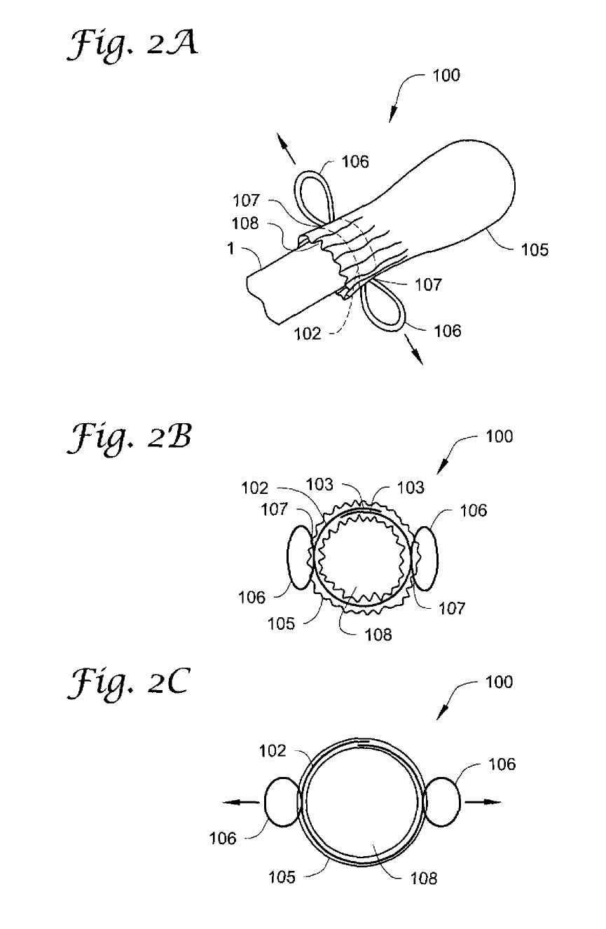

That is, when the penis 1 is inserted through the cuff component 12 and the securing member is tightened, the occlusive protrusion 14 can compress the penis. As a result, such compression can be imparted on the urethra to occlude the urethra and prevent or substantially reduce urine leakage. In one embodiment of operation, FIG. 1B shows the securing member being tightened and retained by the retaining member 16. It will be appreciated that the occlusive protrusion 14 may not be used for compressing the penis, rather compression of the penis can be accomplished by even circumferential compression by tightening the cuff component 12 alone. This concept is further described in the embodiment of FIGS. 2A to 2C.

Turning back to the securing member of the cuff component 12, in one embodiment the securing member is a draw string or strap-like part. It will be appreciated that the securing member is not limited to the specific structure shown, so long as the securing member can be configured to support an occlusive protrusion and/or be capable of being secured to facilitate compression of the penis and compression of the urethra. In one embodiment, the occlusive protrusion 14 is a single bump or other structure of increased dimension. It will be appreciated that the occlusive protrusion is configured to contact the underside of the penis, so that compression can be imparted on the urethra when it is pressed against the penis. It further will be appreciated that the occlusive protrusion 14 is not limited to the specific structure shown, so long as the occlusive protrusion 14 can provide the compressive functionality when pressed against the penis 1.

In one embodiment, the retaining member 16 is a retaining buckle or ring. It will be appreciated that the retaining member 16 is not limited to the specific structure shown, so long as the retaining member 16 can facilitate holding the cuff component 12 and particularly portions proximate the free ends 13 of the securing member.

Regarding the absorbent component 15, any inadvertent urine leakage can be captured and retained by the absorbent component 15. The absorbent component 15 may be constructed as an absorbent sleeve, pocket, shield or pad which is integral to, or detachable from, the cuff component 12. It will be appreciated that the absorbent component 15 can be attached, either integrally or removably, in a variety of ways as one skilled in the art can employ to accomplish such function, and is not limited to the specific configuration shown.

When the absorbent component 15 is integral with the cuff component, 12, such a configuration would allow a user to dispose of the entire unit or clean it and reuse it. When the absorbent component 15 is releasably attached from the cuff component 12, such a configuration 15A would allow a user the option to re-use the cuff component 12 and dispose of the used detachable absorbent component 15.

As shown, the securing member of the cuff component 12 along with the absorbent component 15 form the open region 18 circumferentially, so that when the retaining member 16 is pushed down along the free ends 13, a circumferential compression of the penis 1 occurs. In one embodiment, the retaining member 16 holds together the free ends 13 and portions proximate the free ends 13. In such a configuration, the cuff component 12 is in its occlusive condition, such as by a frictional fit with the retaining member 16.

In one embodiment, the cuff component 12 may be disposed within the absorbent component 15 (as shown by phantom line of FIG. 1A), such that the securing member of the cuff component 12 acts as a draw string. The absorbent component 15 can include slots 17 that allow access to the free ends 13 of the cuff component 12 and allow the securing member to be pulled from the slots 17 for tightening. The slots 17 allow the free ends 13 to be pulled through the absorbent component 15. As shown, the free ends 13 pass through a collapsible portion of an absorbent component 15 surrounding the penis 1. It will be appreciated that at least part of the absorbent component is constructed of a collapsible material at least with respect to the part where the cuff component 12 is to be operated. It will further be appreciated that the entire absorbent component may be constructed as a collapsible material. As the free ends 13 compress the penis 1, the absorbent component 15 may be pulled tightly, so as to allow the occlusive protrusion 14 to press against the penis 1 and impart compression on the urethra to prevent urine leakage.

During tightening of the securing member, the occlusive protrusion 14 can be pressed or pushed against the base of the penis 1 to localize compression of the urethra and stop urine leakage. The occlusive protrusion 14 may be disposed within the absorbent component 15, or the absorbent component may include an opening corresponding to a position where the occlusive protrusion should be placed. It will be appreciated that the occlusive protrusion may be disposed on the absorbent component, rather than on the securing member of the cuff component 12.

In the compression position, the absorbent component 15 surrounds the penis 1 and can provide a fluid tight barrier between inner and outer surfaces of the absorbent component 15. As an example, the absorbent component 15 includes an absorbent material, which can absorb leaked urine to prevent spillage and to maintain a dry penile surface. As some examples only, the absorbent component 15 may include absorbent and liquid barrier layers. The absorbent layer may be constructed of materials including but not limited to woven or non-woven constructions of cotton, other cellulosics, nylon, polyester, polyethylene or polypropylene materials. The liquid barrier layer may be constructed of materials including but not limited to woven or non-woven nylon, polyester, polyethylene or polypropylene materials. It will be appreciated that the absorbent component 15 may be composed of a variety of material, so long as the material accomplishes the function of absorbing inadvertent leakage and being retained with the cuff component 12 when in use.

FIGS. 2A through 2C illustrate another embodiment of a penile compression device 100. For example, occlusion of the urethra is accomplished by an even circumferential compression of the penis 1 and without the use of an occlusive protrusion. It will be appreciated, however, that an occlusive protrusion may be employed but it is not required. FIGS. 2A through 2C show an absorbent component 105 integrally attached with a cuff component 102. The cuff component 102 is a securing member which can be a biasing member, which is normally biased in a contracted mode. As one example the cuff component 102 is an elastomeric band which normally rests in a contracted mode.

Members 106 are attached to the cuff component 102. By manually retracting the members 106 or pulling them away, the cuff component 202 can be expanded from its free ends 103. As a result, the cuff component 102 is expanded from its normally contracted mode to allow insertion of the penis 1 through open region 108 defined by the cuff component 102 and the absorbent component 105.

As shown, portions of the free ends 103 overlap in the contracted mode, where in the expanded mode the free ends 103 have less overlap. In one embodiment, retracting the members 106 allows the free ends 103 of cuff component 102 to slide over one another to increase the circumference of the open region 108 and allow insertion of the penis 1. Releasing the members 106 allows the cuff component 102 to return toward its normal contracted shape under its intrinsic biasing force. It will be appreciated that the amount of overlap of the free ends 103 may be modified as necessary or suitable, so long as the cuff component 102 can be expanded to allow penis insertion therethrough. It further will be appreciated that the cuff component 102 may be a closed loop rather than including the free ends 103, where the cuff component 102 is still constructed to be expandable to allow penis insertion therethrough.

In further operation when penile compression is desired, releasing the members 106 allows the cuff component 102 to collapse onto the penis 1 and bias back toward its initial contracted position. As a result, the penis can be compressed circumferentially to impart compression on the urethra and occlude the urethra and prevent urine leakage. Further manual retraction of the members 106 once again releases penile compression of the cuff component 102, and allows the device 100 to be removed, including the cuff component 102 and the absorbent component 105.

In one embodiment, the members 106 are constructed as pull tabs or loops. As shown, two members 106 are employed. It will be appreciated that the number of members 106 is non-limiting, as one or more than two members could be employed as desired and/or necessary. It further will be appreciated that the members are not limited to the specific structure shown. It further will be recognized that a variety of modifications may be made by one of skill in the art, so long as the resulting member structure can accomplish the function of expanding and contracting the cuff component 102.

The absorbent component 105 can be similarly constructed as described. The absorbent component 105 may be further modified from the absorbent component 15, where slots or openings 107 are also disposed in side positions of the absorbent component 105, so as to be suitable for operating the members 106.

Any of the penile compression devices described herein can be manufactured by a variety of automated, high volume processes, and employing concepts such as those used to produce diapers and absorbent pads. As some illustrative examples, the securing member of the cuff component may be manufactured from woven or non-woven constructions of cotton, nylon polyester, polyethylene, or polypropylene materials. Further, the members may be manufactured, for example, from thermoplastic or thermosetting materials which are injection or transfer molded, or die cut from sheets of these base materials. As described, the absorbent component may include both absorbent and liquid barrier layers. Examples of materials for the absorbent layer may include woven or non-woven constructions of cotton, other cellulosics, nylon, polyester, polyethylene or polypropylene materials. Examples of materials for the liquid barrier may include a construction of woven or non-woven nylon, polyester, polyethylene or polypropylene materials.

In further examples, when the cuff component is an elastomeric band, the cuff component may be manufactured from various elastomeric materials including but not limited to silicone, latex and neoprene rubbers, polyurethane, or other thermoplastic elastomers. In yet other examples, the elastomeric band may be replaced by a circumferential biased metal, such as but not limited to, stainless steel, nickel titanium alloy or rigid thermoplastic material.

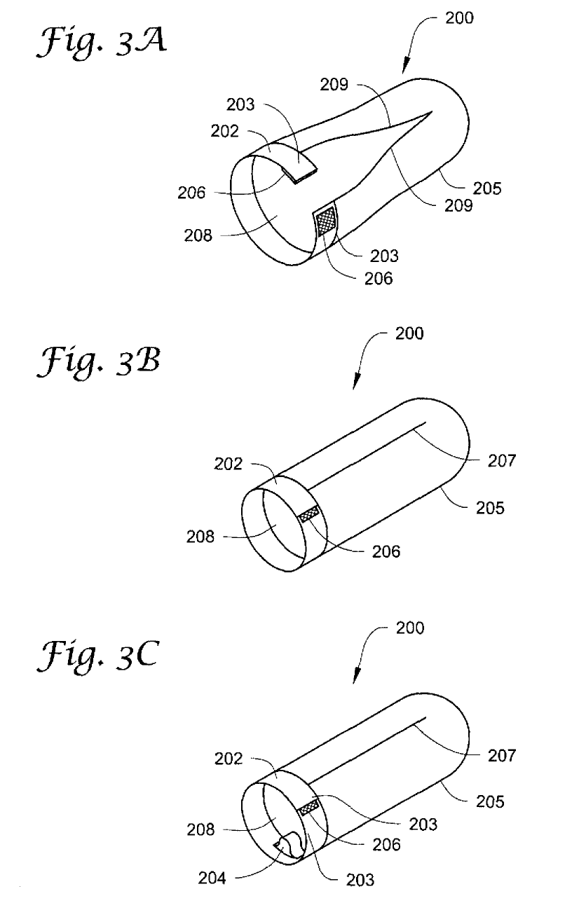

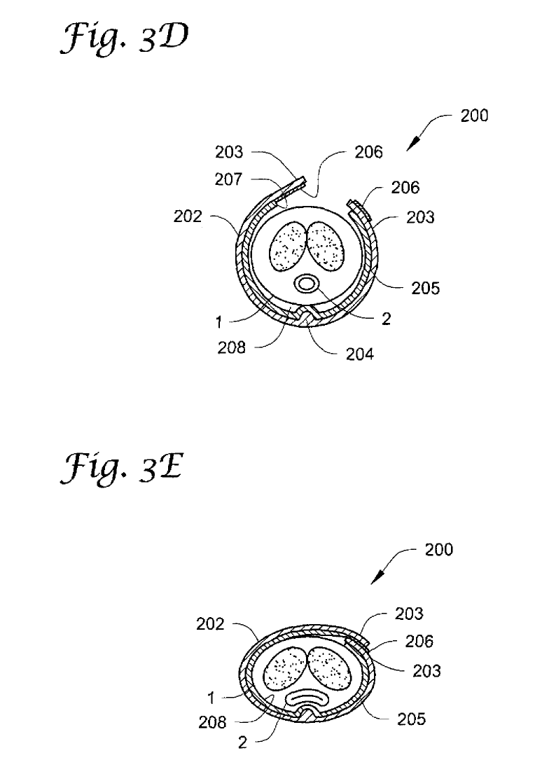

FIGS. 3A through 3E illustrate another embodiment of a penile compression device 200. The penile compression device 200 includes a cuff component 202 and an absorbent component 205. The cuff component 202 includes a securing member that may be a flexible, non-distensible belt or strap-like part. The cuff component 202 can be used to apply circumferential pressure to the penis when it is closed.

FIGS. 3A and 3B show one embodiment, in which the cuff component 202 can be closed so that it is wrapped around the penis. The cuff component 202 can be closed at free ends 203 using retaining members 206 to hold the cuff component in a closed position. In this position, the cuff component 202 can be used to manually apply an external compression on the penis to circumferentially compress the penis, and thereby impart compression upon the urethra. As described, the cuff component 202 may be a belt or strap-like member attached to the absorbent component 205. FIG. 3B shows manual tensioning of the cuff component 202 by closing the free ends 203 using the retaining members 206 to compress the penis and limit urine leakage.

In one embodiment, the retaining members 206 may be configured in a variety of ways, such as a material employing Velcro®, or a button, or a mechanical snap closure, or the like. It will be appreciated that the retaining members 206 are not limited to the specific structure shown, so long as the free ends 203 are configured to close the cuff component 202 at the open region 208.

The absorbent component 205 is attached to the cuff component 202. It will be appreciated that the absorbent component 205 may be integrally or detachably connected to the cuff component 202. In one embodiment, the absorbent component 205 includes free portions 209 along one longitudinal axis, such that the absorbent component 205 can be opened from the side. As shown, the free portions extend proximate the open region 208 from one end, and terminate before reaching the opposite end. In such a configuration, the absorbent component 205 can allow the penis to be easily placed within the absorbent component 205 without the need to insert it first through a closed loop.

In one embodiment, the free portions 209 of the absorbent component 205 can come together or can overlap each other forming a seam 207. The amount of overlap between the free portions 209 of the absorbent component 205 and/or the free ends 203 of the cuff component 202 may vary as necessary and/or desirable. As one example, the position or seam where the free portions 209 of the absorbent component 205 come together is offset from the position or seam where the free ends 203 of the cuff component 202 come together. In such a configuration, the penile compression device can be better secured. It will be appreciated that the configuration for making the free portions 209 come together, including the specific overlap and/or offset, can be accomplished in a variety of ways. It further will be appreciated that the free portions 209 and seam 207 are not limited to the specific structure shown, so long as the absorbent component can be closed along the side.

In one embodiment, attachment of the absorbent component 205 to the cuff component 202 can be configured, such that manual tensioning of the cuff component 202 pulls one free portion 209 of the absorbent component 205, allowing it to overlap the other free portion 209. In such a configuration, a liquid barrier can be created to prevent leakage.

In operation, FIGS. 3A and 3B the cuff component 202 and absorbent component 205, can further open the open region 208 to allow the penile compression device 200 to wrap around the penis and thereby compress the penis.

FIGS. 3C through 3E show another embodiment of the penile compression device 200 that includes an occlusive protrusion 204. Similarly as occlusive protrusion 14, the occlusive protrusion 204 may be placed on the cuff component to allow concentration of occlusive pressure on the base of the penis 1 to further limit urine leakage through the urethra 2. FIG. 3D shows the penile compression device 200, having the occlusive protrusion 204, in a position before the penis 1 is compressed and the urethra 2 occluded. FIG. 3C shows the device 200 in a compression position, where the device 200 can be used to compress the penis and occlude the urethra. FIG. 3E shows the penis 1 compressed and the urethra 2 occluded using the device 200.

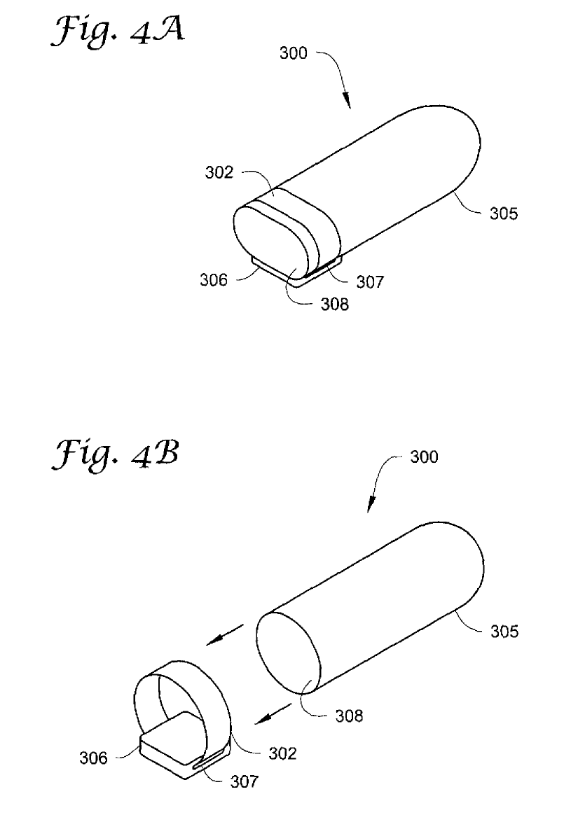

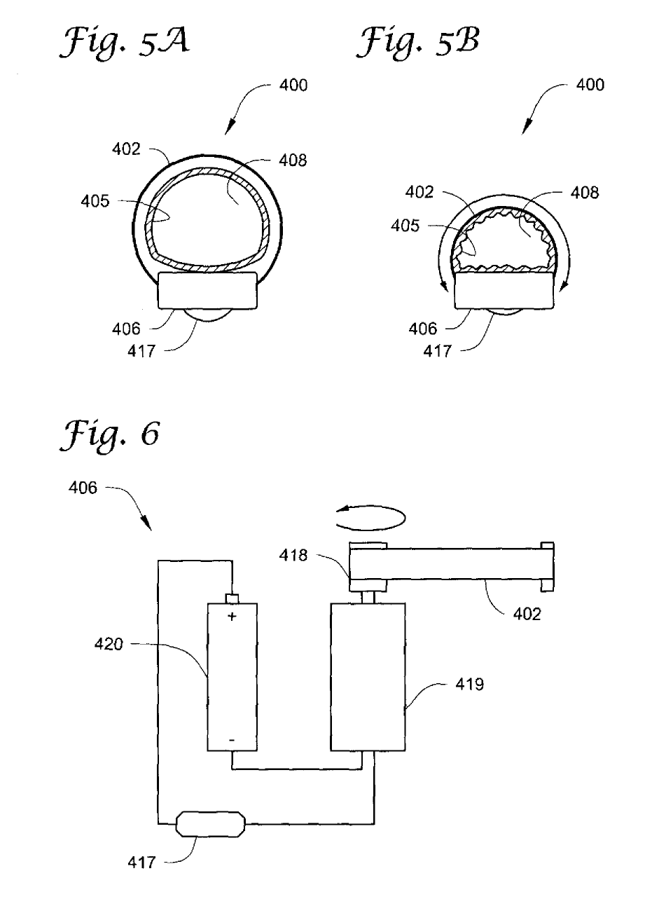

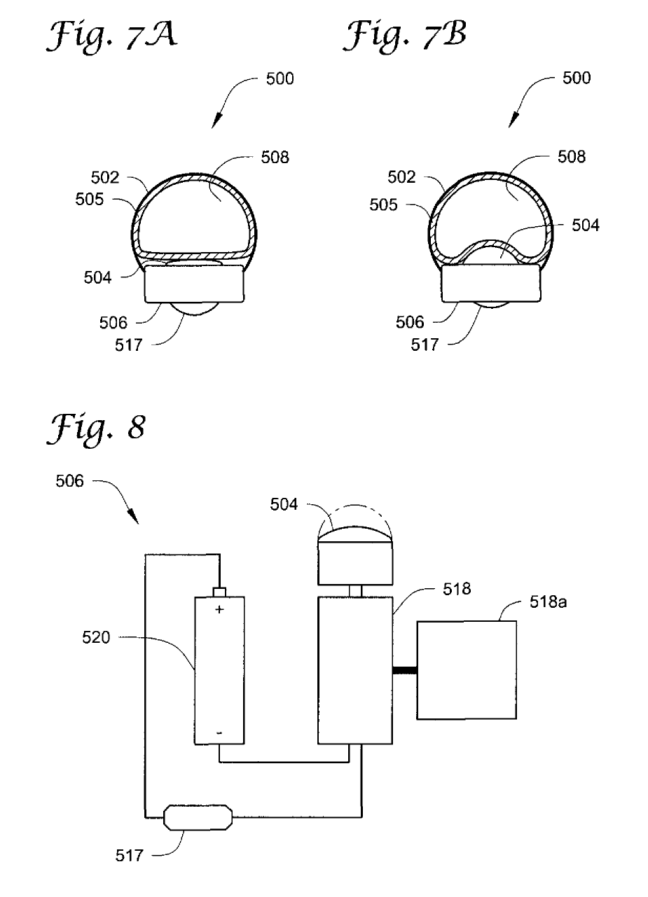

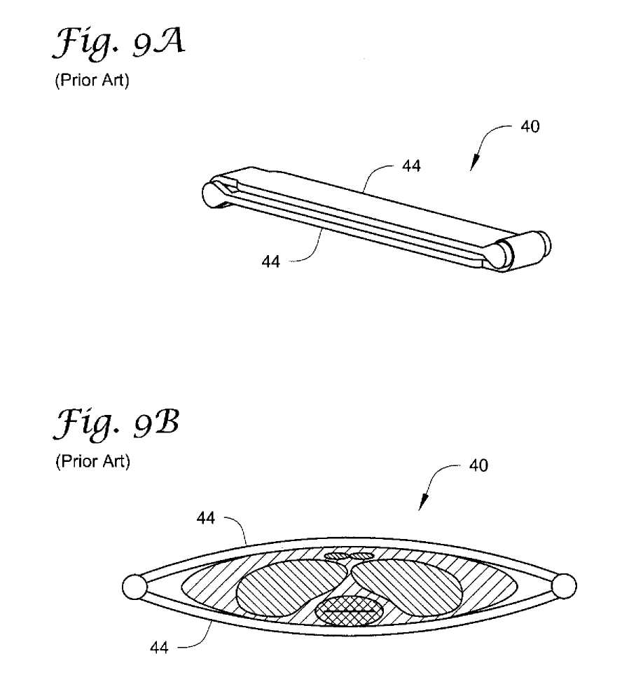

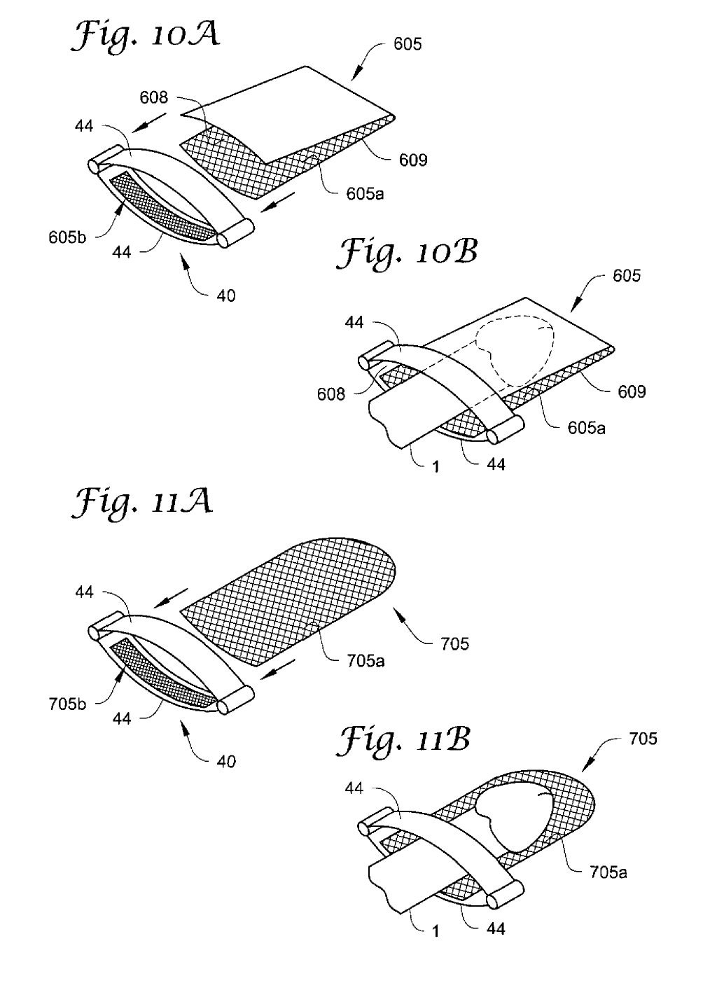

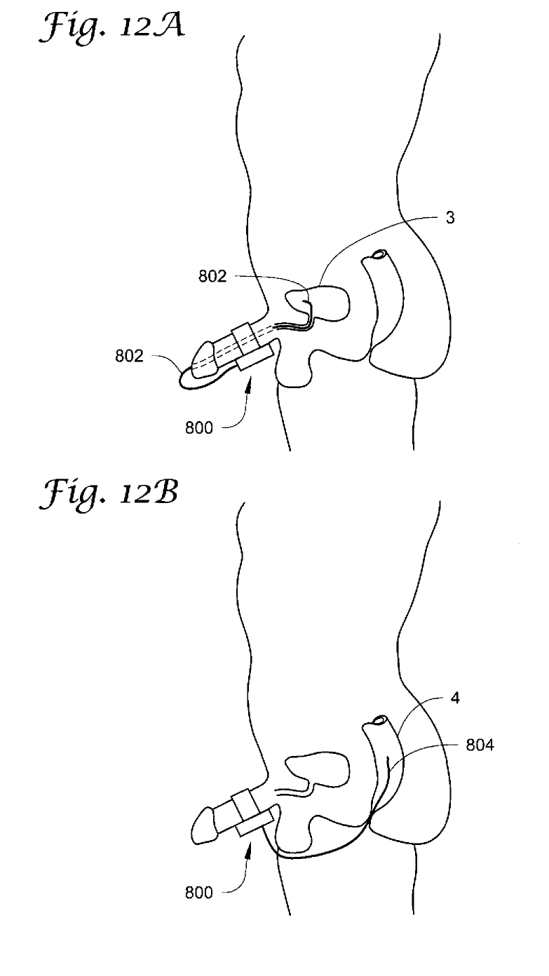

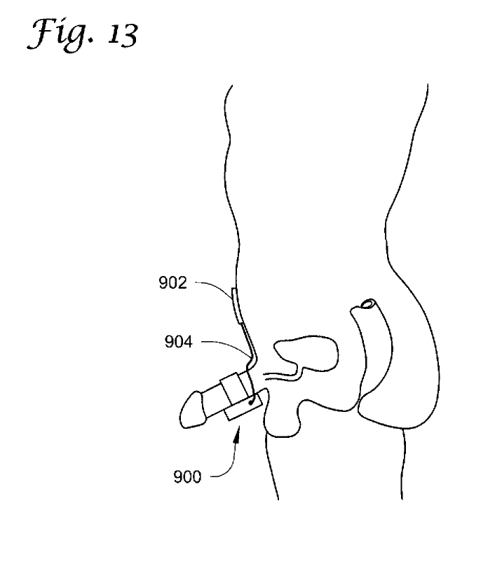

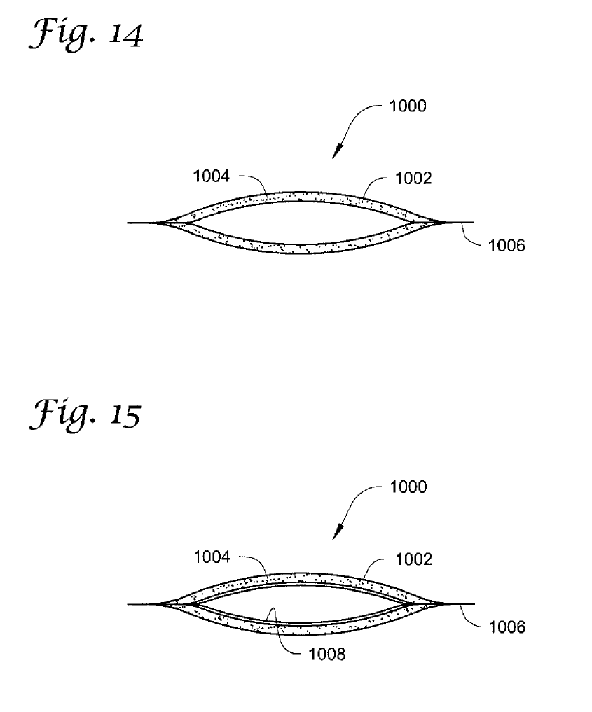

FIGS. 4A and 4B illustrate another embodiment of a penile compression device 300. The penile compression device 300 includes a cuff component 302 and an absorbent component 305.

The cuff component 302 is a securing member that may be a strap or belt-like part. It will be appreciated that the cuff component 302 is not limited to the specific structure shown, and may be constructed a number of ways, so long as it can wrap around a penis and allow a penis to be inserted therethrough.

As shown in FIG. 4B, the absorbent component 305 can be releasably attached with the cuff component 302, and includes an open region 308 at one end. The absorbent component 305 may be constructed as already described so as to be suitable for this embodiment. Further, the absorbent component 305 may be a disposable absorbent part. As some examples, the absorbent component 305 can be temporarily affixed to the cuff component by a variety ways, including but not limited to mechanical, adhesive, or Velcro® attachments, or the like. After use, the absorbent component 305 may be detached and disposed, while the cuff component 302 may be reused with another absorbent component that would replace the one used before.

The penile compression device 300 also includes a reusable compression mechanism 306. The compression mechanism 306 is configured to compress the penis and thereby impart compression on the urethra to provide an occlusive effect. In this regard, the compression mechanism 306 takes the place of and functions similar to the retaining members or occlusive protrusion previously described. In some embodiments, the compression mechanism 306 may be suited for use as an electromechanical or electro-hydraulic compression mechanism, and includes a housing that contains the retaining member or occluding components. FIGS. 5A through 8 below further describe such components.

The compression mechanism 306 also includes slots 307 in its housing so that the cuff component 302 can connect with the compression mechanism 306. It will be appreciated that the cuff component 302 can be connected to the compression mechanism 306 by a number of ways. It further will be appreciated that the connection between the cuff component 302 and the compression mechanism 306 is not limited to the specific structure shown, so long as the connection allows cooperation of the compression mechanism 306 and the cuff component 302 to achieve urethral occlusion.

FIGS. 5A through 6 illustrate one embodiment of a penile compression device 400. The penile compression device 400 includes a cuff component 402 and an absorbent component 405. The absorbent component 405 may be constructed as already described, and its construction is not further discussed. The cuff component 402 and the absorbent component 405 are configured to define an open region 408. The open region 408 allows for the penis to be inserted through the cuff component 402 and into the absorbent component 405. The cuff component 402 is connected with a compression mechanism 406.

As shown, the compression mechanism 406 is an electromechanical compression mechanism. In one embodiment, the cuff component 402 is a flexible, non-distensible belt or strap-like member, which can surround the penis. When compression is desired, the cuff component 402 is drawn within a housing of the compression mechanism 406 by operating an actuator 417. In one embodiment, the actuator 417 is a switch. It will be appreciated that the actuator 417 is not limited to the specific structure shown, so long as the actuator 417 can function to operate the compression mechanism 406 to draw the cuff component 402 into the housing of the compression mechanism 406.

When the compression mechanism 406 is operated to draw the cuff component 402 into the housing, the cuff component 402 can apply an amount of compression on the penis. In some examples, the amount of compression applied can be either a pre-determined amount of compression on the penis or an amount of compression determined by the user. As a result, the amount of compression applied can impart compression on the urethra and provide the occlusive effect to prevent urinary leakage or at least substantially reduce it. In one embodiment, the compression applied is an even circumferential compression, which tightens the cuff component 402 on the penis.

In FIG. 6, the actuator 417 is controlled by electromechanical components. In one example, the compression mechanism 406 includes electromechanical components that would reside in a housing. The electromechanical components include a take-up component 418 connected to the cuff component 402. It will be appreciated that slots may be employed as described in FIGS. 4A and 4B, so that the cuff component 402 can be connected to the compression mechanism 406. The take-up component 418 is further connected to a motor component 419, which is powered using a power supply 420.

Pushing the actuator a first time can activate the motor component 419 and move the take-up component 418 to draw the cuff component 402 into the housing of the compression mechanism 406. Pushing the actuator 417 a second time reverses the motor component 419 and take-up component 418 to remove compression from the penis and occlusive pressure on the urethra. It will be appreciated that the actuator 417 may be single element, such as a button, switch, or the like, and that such single element is operable for putting the cuff component in the compressive and non-compressive positions. It will further be appreciated that the actuator 417 may comprise multiple elements to carry out putting the cuff component in the compressive and non-compressive positions if necessary or desired.

As an example, the take-up component 418 is a spool, while the motor component 419 is an electric motor. In one embodiment, the power supply 420 is a battery. It will be appreciated that the components of the compression mechanism 406 are not limited to the specific structures shown. It further will be appreciated that the electromechanical components of the compression mechanism 406 can be accomplished in a variety of ways, so long as the compression mechanism 406 is capable of providing the amount of compression suitable to give the occlusive effect on the urethra.

FIGS. 7A through 8 illustrate another embodiment of a penile compression device 500. The penile compression device 500 includes a cuff component 502 and an absorbent component 505. The absorbent component 405 may be constructed as already described, and its construction is not further discussed. The cuff component 502 and the absorbent component 505 are configured to define an open region 508. The open region 508 allows for the penis to be inserted through the cuff component 502 and into the absorbent component 505. The cuff component 502 is connected with a compression mechanism 506. As shown, the compression mechanism 506 is an electro-hydraulic compression mechanism.

As in FIGS. 5A and 5B, the cuff component 502 can be a flexible, non-distensible belt or strap-like member, which can surround the penis. When compression is desired, an occlusive member 504 is actuated from a housing of the compression mechanism 506. In one embodiment, the occlusive member 504 is expandable when it is actuated and can press against the penis to provide a desired occlusive effect on the urethra. The occlusive member 504 is actuated by operating an actuator 517. In one embodiment, the actuator 517 is a switch. It will be appreciated that the actuator 517 is not limited to the specific structure shown, so long as the actuator 517 can function to activate the occlusive member 504 from the housing of the compression mechanism 506.

When the compression mechanism 506 is operated, the occlusive member 504 is activated from the housing and can apply an amount of compression on the penis, which thereby imparts localized occlusion of the urethra. In some examples, the amount of compression applied by the occlusive member 504 can be either a pre-determined amount of compression on the penis or an amount of compression determined by the user. As a result, the amount of compression applied can impart compression on the urethra to provide the occlusive effect and prevent urinary leakage or at least substantially reduce it. It will be appreciated that an occlusive member may not be employed and that the entire cuff component may be configured to be expanded if desired or necessary. In such a configuration, urethral occlusion may be achieved through an even circumferential compression, rather than by localized compression.

In FIG. 8, the actuator 517 is controlled by electro-hydraulic components residing within the housing of the compression mechanism 506. In one example, the electro-hydraulic components are disposed in the housing of the compression mechanism 506 and/or on its housing. The components include the occlusive member 504, which may be disposed within or on the housing, or a combination of both. It will be appreciated that, to the extent the occlusive member 504 is disposed within the housing, the housing would include an opening so that the occlusive member 504 can be released or expanded from the housing. The opening is not shown, however, one of skill in the art would be able to construct the opening in any number of ways that would be suitable to allow the occlusive member to be expanded. As with FIGS. 5A and 5B, it further will be recognized that slots may be employed, so that the cuff component 502 is connected to the compression mechanism 506.

The occlusive member 504 is further connected to an expanding component 518, which is powered using a power supply 520. Pushing the actuator 517 a first time can activate the expanding component 518 and expand the occlusive member 504 to provide the necessary amount of compression on the penis. Pushing the actuator 517 a second time reverses the expanding component 518, so that the occlusive member 504 can be contracted to remove compression from the penis and occlusive pressure on the urethra.

As with the actuator 417 described in FIGS. 5A-6, the actuator 517 may be single element, such as a button, switch, or the like, and that such single element is operable for putting the cuff component in the compressive and non-compressive positions. It will further be appreciated that the actuator 517 may comprise multiple elements to carry out putting the cuff component in the compressive and non-compressive positions if necessary or desired.

As an example, the occlusive member 504 is a balloon that can be inflated and deflated with fluid to provide the respective occlusion effect and compression release. In one embodiment, the expanding component 518 is a pump, where the power supply 520 is a battery. The pump further includes a fluid reservoir 518a. In operation, depressing the switch a first time can put fluid into the balloon and expand the balloon to provide the necessary amount of compression on the penis. Depressing the switch a second time reverses the pump, pulling fluid out of the balloon and returning it to the fluid reservoir 518a, so as to allow normal urinary flow. In one embodiment, the fluid used for inflation may be water, saline, air, other gas(es), combinations of these fluids, or the like.

It will be appreciated that the components of the compression mechanism 506 are not limited to the specific structures shown. It further will be appreciated that the electro-hydraulic components of the compression mechanism 506 can be accomplished in a variety of ways, so long as the compression mechanism 506 is capable providing the amount of compression suitable to give the occlusive effect on the urethra.

In both electro-mechanical and electro-hydraulic penile compression devices described, additional electrical hardware and software may be added to allow any of the devices to sense sudden increases in abdominal pressure or abdominal muscular activity such as may cause or indicate the potential for urine leakage. Once sensed, the devices would be activated to provide a momentary occlusion of the urethra to prevent urinary leakage. This momentary rather than constant occlusion can maximize user comfort and minimize potential damage to the compressed organ, such that the device would be activated when it is actually needed. That is, occlusion and non-occlusion can be automatically controlled.

As one example, sensing of abdominal pressures could be accomplished through use of intra-abdominal pressure transducers introduced into the bladder, rectum, or surgically implanted into the abdomen. FIGS. 12A and 12B illustrate one embodiment of a pressure transducers 802, 804, which may be incorporated with a penile compression device 800. As one example, the penile compression device 800 may be either of the electro-mechanical and electro-hydraulic penile compression devices described above. FIG. 12A shows the pressure transducer 802 connected to the penile compression device 800, where the pressure transducer 802 is a catheter based in the bladder 3 to sense pressure, such as may cause or indicate the potential for urine leakage. FIG. 12B shows the pressure transducer 804 connected to the penile compression device 800, where the pressure transducer is a catheter based in the rectum 4 for measuring intra-abdominal pressures.

As another example, sensing muscular activity can be accomplished with skin electrodes used to sense electromyographic signals through the skin. FIG. 13 illustrates one embodiment of a skin electrode 902 connected to a penile compression device 900 through a cable 904. As with the pressure transducers, the skin electrode 902 may be incorporated with a penile compression device, such as either of the electro-mechanical and electro-hydraulic penile compression devices described above. In one embodiment, the skin electrode is an electromyographic electrode that can sense abdominal muscular activity, such as may cause or indicate the potential for urine leakage.

It will be appreciated that introduction of such sensory or monitoring components, can be achieved by a number of ways that one of skill in the art could employ.

Turning back to the absorbent component, the absorbent components described above are depicted to totally envelop the penis to prevent leakage and wicking of urine from the absorbent material to the user's undergarments. However, reduced urine leakage due to the improved penile compression devices described may make possible that an absorbent structure is not required to totally envelop the penis. That is, an acceptable option can be that an absorbent component may be constructed which does not totally envelop the penis.

Two different non-enveloping absorbent components 605, 705 are shown below in FIGS. 10A-11B and may be employed in with any of the penile compression devices described, so long as such use is consistent with the particular embodiment discussed.

Before describing the improved absorbent components 605, 705, GT Urological, LLC, the Assignee of the instant application, also discloses a penile compression clamp in its U.S. patent application Ser. No. 11/122,395 for “Penile Compression Device,” which is now published U.S. Patent Application Publication No. 2005-0256365 having the same title and mentioned above. FIGS. 9A and 9B illustrate an embodiment of a penile compression clamp 40 disclosed in this application, for which the non-enveloping absorbent components 605, 705 are shown attached as in FIGS. 10A through 11B.

It is recognized that the non-enveloping absorbent components 605, 705 are shown attached to the arms 44 of the penile compression clamp 40 for exemplary purposes only. As described above, it further is recognized that the non-enveloping absorbent components 605, 705 may be employed with any of the above detailed penile compression devices, so long as such incorporation is consistent with the inventive concept of the particular embodiment.

In one embodiment, the non-enveloping absorbent components 605, 705 require fewer manufacturing steps than a totally enveloping design and lend themselves to high volume manufacturing procedures. FIG. 10A and 10B depict an absorbent component 605 wrapping around the end of the penis 1 to enhance absorption. The absorbent component 605 is then permanently or removably affixed to two surfaces on the arms 44 of the clamp 40. That is, free ends of the absorbent component 605 can be attached to the arms 44, 44 of the clamp 40. In one embodiment, a strip 605b may be disposed on a location of a cuff component to indicate a location where the absorbent component 605 can be attached. In some examples, the strip 605b may be a piece of Velcro® or adhesive sufficient to attach the absorbent component 605 to a cuff component. In such a configuration, one free end is attached to one arm and the other free end is attached to the other arm. The absorbent component 605 includes an absorbent material 605a and the free ends include an open region 608, where the penis can be inserted. The absorbent component 605 can wrap around the end of the penis but has open sides 609. A fold is present at one end of the absorbent component. As an example, the fold is disposed at an end opposite the end where the open region 608 resides.

FIGS. 11A and 11B depict how an absorbent component 705 may be affixed permanently or removably to one surface an arm 44 of the clamp 40. That is a free end of the absorbent component 705 can be attached to one of the arms 44 of the clamp 40. As with the absorbent component 605 in FIGS. 10A and 10B, a strip 705b may be disposed on a location of a cuff component to indicate a location where the absorbent component 705 can be attached. In some examples, the strip 705b may be a piece of Velcro® or adhesive sufficient to attach the absorbent component 705 to a cuff component. As with absorbent component 605, the absorbent component 705 includes an absorbent material 705a. As shown, the absorbent component 705 can be one surface that can contact one side of the penis 1, rather than two surfaces as in the absorbent component 605.

Turning back to the absorbent component structure, FIGS. 14 and 15 illustrate examples of absorbent component constructions in accordance with the inventive concepts herein and that may be employed with any of the embodiments described. FIG. 14 show one configuration of an absorbent component 1000. The absorbent component 1000 may be constructed with a liquid barrier layer 1002 and a liquid absorbent layer 1004. Edges 1006 may be sealed to provide the generally sleeve-like configuration. In one embodiment, the edges may be heat sealed. However, it will be appreciated that the edges may be sealed by employing various approaches as known and suitable in the art, so long as the edges are connected together.

As discussed above, the absorbent layer may be constructed of materials including but not limited to woven or non-woven constructions of cotton, cotton batting, other cellulosics, nylon, polyester, polyethylene or polypropylene materials. The liquid barrier layer may be constructed of materials including but not limited to woven or non-woven nylon, polyester, polyurethane, polyethylene, polypropylene, or silicon materials. It will be appreciated that the absorbent component 1000 may be composed of a variety of material, so long as the material accomplishes the function of absorbing inadvertent leakage and being retained with the cuff component when in use.

FIG. 15 illustrates that the absorbent component 1000 may further include a transitional layer 1008. The transitional layer 1008 is disposed as the innermost layer that would contact the penis, and can be functional for allowing wicking of urine away from the penis into the absorbent layer. Such a transitional layer 1008 can help maintain dryness of the penile skin. In some examples, the transitional layer may be a woven or non-woven polyethylene, polypropylene, or cotton.

It will be appreciated that any of the components of the penile compression devices in FIGS. 1A-8 and 10A-11B described and their inventive concepts may be combined or not combined, so long as such combination is not inconsistent with the embodiment(s) disclosed. For example, any of the penile compression devices may employ an occlusive protrusion or occlusive member as necessary or desired to provide suitable localized urethral occlusion. As another example, slots may be employed in any of the absorbent components so that a cuff component may be disposed within layers of the absorbent component. As yet another example, any of the embodiments described may employ an absorbent component configured to be integrally or removably attached with the cuff component, and which may or may not totally envelop the penis when used. It will be appreciated that these examples are non-limiting and are described for exemplary purposes to illustrate that the inventive concepts of many of the embodiments can be combined.

It further will be appreciated that the penile compression device can be universally fitting, so as to be used on a variety of penile sizes and dimensions. That is, the penile compression devices described herein can be modified as necessary and suitable to fit any penis. Any of the penile compression devices described are suitably constructed to provide sufficient pressure on the penis to impart compression of the urethra. In one embodiment, penile compression can be achieved with a pressure in the range of 0.500 kg to 0.850 kg.

The above specification provides a complete description of the composition, manufacture and use of the improved penile compression device in accordance with the principles of the inventive concepts disclosed. Since many embodiments of the penile compression device can be made without departing from its spirit and scope, the invention resides in the claims hereinafter appended.

We claim:

1. A penile compression device comprising:
a cuff component; and
an absorbent component attached to the cuff component,
the cuff component being adapted to compress a penis and to impart compression on the urethra of the penis,
the cuff component being adapted to press a portion of the absorbent component against the penis when the cuff component compresses the penis,
the cuff component being adapted to occlude the urethra when the cuff component compresses the penis,
wherein the cuff component and the absorbent component prevent urine leakage and spillage from the urethra and penis.

2. The device of claim 1, wherein the cuff component being adapted to surround the penis and to apply an even circumferential compression on the penis.

3. The device of claim 1, wherein the cuff component comprising an occlusive protrusion disposed on one surface of the cuff component, the occlusive protrusion adapted to apply a localized compression on the penis, and when the cuff component compresses the penis, the occlusive protrusion being disposed corresponding to a position that faces and imparts compression on the urethra.

4. The device of claim 3, wherein the occlusive protrusion being configured as a single bump structure.

5. The device of claim 3, further comprising an electro-hydraulic compression mechanism, the occlusive protrusion is an expandable member being adapted to expand when the hydraulic compression mechanism is activated, the occlusive protrusion being adapted to compress the penis and to impart compression on the urethra.

6. The device of claim 1, wherein the cuff component is a flexible, distensible securing member.

7. The device of claim 6, wherein the securing member being adapted to compress the penis when the securing member is manually tightened by a user, the securing member being configured to be manually tightened with a retaining member.

8. The device of claim 6, further comprising an electromechanical compression mechanism, the securing member being adapted to compress the penis when the electro-mechanical compression mechanism is activated to draw the securing member into a housing of the electro-mechanical compression mechanism.

9. The device of claim 1, wherein the cuff component is a flexible, distensible securing member, the securing member being configured as a biasing member that is intrinsically biased toward a compressive position, the biasing member is configured to be expanded in a non-compressive position so as to be adapted to allow placement on or removal from the penis, the biasing member being configured to return to the compressive position so as to be adapted to allow placement on and compression of the penis.

10. The device of claim 1, wherein the absorbent component is configured to removably attached with the cuff component or permanently attached with the cuff component.

11. The device of claim 1, wherein the absorbent component is constructed and arranged with two surfaces that are attached to the cuff component, the two surfaces contact sides of the penis when the cuff component is applied to compress the penis, the two surfaces include a fold therebetween, such that the absorbent component being adapted to wrap around the head of the penis and to allow a portion of the penis to not be wrapped by the absorbent component.

12. The device of claim 1, wherein the absorbent component is constructed and arranged with a single surface that is attached to the cuff component, the single surface contacts a side of the penis when the cuff component is applied to compress the penis, and the absorbent component being adapted to allow a portion of the penis to not be wrapped by the absorbent component.

13. The device of claim 1, wherein the absorbent component including free portions longitudinally extending from an open region toward a closed end, the free portions terminating proximate the closed end, and the free portions being adapted to allow the absorbent component receive the penis from a side of the absorbent component.

14. The device of claim 1, further comprising a pressure transducer connected with the penile compression device, the pressure transducer configured to be connected to a body of a user and configured to sense abdominal pressure that indicates a potential for urine leakage.

15. The device of claim 14, wherein the pressure transducer is configured to be present in at least one of the bladder, rectum, and abdomen.

16. The device of claim 1, further comprising a skin electrode connected with the penile compression device, the skin electrode configured to be connected to a body of a user and configured to sense abdominal muscular activity that indicates a potential for urine leakage.

17. A method of externally compressing a penis comprising:
applying a cuff component onto a penis;
applying an absorbent component that is attached to the cuff component onto the penis;
the step of applying the cuff component onto the penis includes compressing the penis and imparting compression on the urethra of the penis,
the step of applying the cuff component and the absorbent component onto the penis includes pressing a portion of the absorbent component against the penis when the cuff component compresses the penis, and
occluding the urethra when the cuff component imparts a compression on the urethra,
wherein compressing the penis and occluding the urethra prevents urine leakage and spillage from the penis and urethra.

18. The method of claim 17, wherein the step of applying the cuff component on the penis includes surrounding the penis and the step of compressing the penis includes applying an even circumferential compression on the penis.

19. The method of claim 17, wherein the step of applying the cuff component includes applying an occlusive protrusion onto the penis, the step of applying an occlusive protrusion includes applying a localized compression on the penis and imparting compression on the urethra.

20. The method of claim 19, wherein the step of applying an occlusive protrusion onto the penis includes expanding the occlusive member to compress the penis and to impart compression on the urethra.

21. The method of claim 17, wherein the step of applying the cuff component onto the penis includes one of manually tightening the cuff component or electromechanically tightening the cuff component.

22. The method of claim 17, wherein the step of applying cuff component onto the penis includes expanding a flexible, distensible securing member from an intrinsically contracted compressive position, to an expanded non-compressive position so as to be adapted to allow placement on or removal of the cuff component from the penis, and returning the cuff component to the compressive position so as to be adapted to allow compression of the penis.

23. The method of claim 17, further comprising sensing at least one of increases in abdominal pressure or abdominal muscular activity to determine whether to activate the cuff component into a compression position.

24. An absorbent component for use with a penile compression device comprising:
a sheet having inner and outer surfaces;
the inner surface being an absorbent material and the outer surface being a liquid barrier material,
the sheet being adapted to be removably attached or permanently attached to a cuff component of the penile compression device,
the sheet being constructed and arranged as a single surface or as two surfaces adapted to contact sides of a penis.

25. The component of claim 24, wherein the single surface being adapted to allow a portion of the penis to not be wrapped by the absorbent component.

26. The component of claim 24, wherein the two surfaces include a fold therebetween, such that the sheet being adapted to wrap around the head of the penis and to allow a portion of the penis to not be wrapped by the absorbent component.

27. The component of claim 24, the sheet is adapted to be wrapped around the penis and comprises free portions longitudinally extending from an open region toward a closed end, the free portions terminating proximate the closed end, and the free portions being adapted to allow the sheet to receive the penis through a side of the sheet.
